(19) United States
(12) Patent Application Publication
(10) Pub. No.: US 2018/0356343 A1
(43) Pub. Date: Dec. 13, 2018

(54) FLUORESCENT NANODIAMONDS AS FIDUCIAL MARKERS FOR MICROSCOPY AND FLUORESCENCE IMAGING

(71) Applicant: THE UNITED STATES OF AMERICA, AS REPRESENTED BY THE SECRETARY, DEPARTMENT OF HEALTH AND HUMAN SERV, Bethesda, MD (US)

(72) Inventors: Keir Cajal Neuman, Bethesda, MD (US); Han Wen, Silver Spring, MD (US); Jennifer Hong, Loredo, TX (US); Chang Kuyn Yi, Germantown, MD (US); Ambika Bumb, Greer, SC (US); Susanta Kumar Sarkar, Rockville, MD (US); Asit Kumar Manna, Rockville, MD (US); Lawrence Elliot Samelson, Chevy Chase, MD (US)

(21) Appl. No.: 15/780,388

(22) PCT Filed: Dec. 2, 2016
(86) PCT No.: PCT/US2016/064523

§ 371 Date: May 31, 2018

Related U.S. Application Data
(60) Provisional application No. 62/262,058, filed on Dec. 2, 2015.

(51) Int. Cl. G01N 21/64; G01N 33/533

(52) U.S. Cl. CPC G01N 21/6428; G01N 2021/6439; G01N 33/533

(57) ABSTRACT

Fiducial marker compositions comprising fluorescent nanodiamonds and methods of making and using the fiducial marker compositions are disclosed. The fiducial marker composition comprises a substrate, and a fluorescent nanodiamond immobilized on a surface of the substrate, wherein the substrate and immobilized fluorescent nanodiamond are optionally top coated with an inert top coating. The fiducial marker compositions are used in imaging methods to correct for drift and other alignment instabilities, and are particularly useful in super-resolution imaging.

### CROSS REFERENCE TO RELATED APPLICATIONS

This application claims the benefit of U.S. Provisional Patent Application Ser. No. 62/262,058, filed Dec. 2, 2015, incorporated herein by reference in its entirety.

### BACKGROUND

Fiducial markers provide stable fixed points on a slide or sample in various types of imaging systems. All measurements are referenced to these points to eliminate sample drift.

Existing fiducial markers are based on gold nanoparticles, fluorescently labeled nm-scale beads, and less commonly quantum dots. All three of these fiducial markers have multiple limitations.

Currently the most commonly used fiducial markers are gold nanoparticles, which are commercially available from a number of sources such as Hestzig LLC. In this approach gold nanoparticles are embedded in a glass coverslip. The gold particles exhibit a size- and shape-dependent emission, which does not bleach over time.

However, gold nanoparticle fiducial markers have multiple drawbacks: They have a narrow emission (photoluminescence) wavelength, which is related to size of the particle. It is difficult to control the emission of the gold particles since emission wavelength, emission intensity, and particle size are coupled. It can therefore be difficult to tune emission intensity without changing the emission wavelength. Additionally, gold can exhibit polarization-dependent emission intensity. It is difficult to obtain correlation over multiple wavelengths. Also, gold particles can blink over time.

Dye labeled nm scale beads, e.g., TetraSpeck beads (Life Technologies, Cat. # T7279) are another less frequently used fiducial marker. The advantage is the small size (100 nm) and the incorporation of four different dyes that cover a wide range of emission wavelengths. The crucial limitation of these beads is that they bleach over time, limiting their usefulness for extended imaging experiments.

Quantum dots are much less frequently used as fiducial markers. Although they are bright fluorescent probes, they suffer from blinking, narrow emission wavelengths, their emission intensity is difficult to adjust, and they bleach over long periods of time. Thus, they are often too bright to be used for many applications in which the fluorophores being measured are quite dim, and they are not suitable for tracking applications over extended periods of time.

Despite the advantages of traditional fluorescence microscopy, the technique is hampered in ultrastructural investigations due to the resolution limit set by the diffraction of light, which restricts the amount of information that can be captured with standard objectives. The resolution limit of light microscopy has been surpassed by techniques known collectively as super-resolution microscopy.

A variety of super-resolution microscopy techniques have recently been developed to overcome the diffraction limit of light microscopy, enabling visualization of small molecular structures. Among these, a category of super-resolution techniques called single molecule localization microscopy (SMLM), which includes photo-activation localization microscopy (PALM) and stochastic optical reconstruction microscopy (STORM), allows the highest level of imaging precision (10-20 nm). SMLM techniques have in common fluorescent probes that can be switched between on (fluorescent) and off (dark/photo-switched) states, isolation of fluorescence from single molecules, and sequential localization of Gaussian-fitted fluorescent peaks.

Due to its compatibility with commercial dyes and microscopes, direct STORM (dSTORM) has become a widely adopted SMLM technique. In addition, dSTORM can routinely achieve localization precision of about 10 nm, compared to about 20 nm achieved with PALM. However, despite the high localization precision (typically calculated using Thompson's equation), accurate localization of single molecules using SMLM has been hampered by a number of issues. First, ‘localization precision’ has often been confused with ‘localization accuracy’ (e.g. a Gaussian-fitted peak with 10 nm precision has been incorrectly assumed to be within 10 nm of the true location of the fluorescent probe). This has important consequences in that, localization precision of 5-10 nm (achieved by most dSTORM studies) is not sufficient to accurately localize single molecules with a high degree of confidence. Second, additional localization uncertainty is introduced by microscope stage movement including drift and vibration.

In addition to the diffraction limit, investigators have been restricted by a spectral limit to light microscopy. Simultaneous visualization of multiple molecules requires fluorescent probes with non-overlapping spectral profiles, generally restricting fluorescence microscopy to 6 colors. In SMLM, only a few fluorescent probes have the properties required for precise single molecule localization, limiting most studies to 2-3 colors. Moreover, non-linear chromatic aberration between the color channels add significant uncertainty to alignment of multiplexed images (Pertsinidis, A. et al. (2010) Nature 466(7306): 647-651; Erdelyi, M. et al. (2013) Opt Express 21(9): 10978-10988). To overcome these spectral limits, alternative multiplexing schemes have been devised (Gerdes, M. J. et al. (2013) Proc Natl Acad Sci USA 110(29): 11982-11987; Jungmann, R. et al. (2014) Nat Methods 11(3): 313-318; Schubert, W. (2014) J Mol Recognit 27(1): 3-18). These strategies utilize cycling of pre-labeled fluorescent probes that are bound to proteins of interest within the cell, imaged, and then either photo- or chemical-bleached. Such multiplexing strategies can indeed bypass the spectral limit of microscopy, but the eventual accumulation of fluorescent probes will likely lead to steric blocking of additional binding sites in the cell, preventing further multiplexing. Furthermore, as fluorescence bleaching is known to be a toxic process (Jacobson, K. et al. (2008) Trends Cell Biol 18(9): 443-450), prolonged photo- or chemical-bleaching will likely cause unwanted effects such as reverse cross-linking and denaturation of cellular proteins.

To date, super-resolution imaging has been limited by the performance of available fiducial markers. These limitations are compounded by the long recording times required to sequentially image 10, 20, or more proteins. Imaging of each protein can take longer than 1 hour, and there can be substantial mechanical drift introduced by repeated washes and incubation steps associated with imaging each protein. The ultimate resolution and registration of the images for individual proteins is determined by the stability and accuracy of the fiducial tracking.

Thus, there is a need for improved fiducial markers for imaging applications, such as fluorescence imaging, particularly for extended imaging of a single sample over time periods that can be as long as a week or more.

### SUMMARY

Disclosed herein are fiducial marker compositions comprising fluorescent nanodiamonds (FNDs) and methods for preparation and use of the compositions.

In an embodiment, the fiducial marker composition comprises a substrate, and a fluorescent nanodiamond (FND) immobilized on a surface of the substrate, wherein the substrate and immobilized FND are at least partially top coated with an inert top coating.

In an embodiment, the fiducial marker composition comprises a substrate, a transparent polymer immobilized on a surface of the substrate, and a fluorescent nanodiamond (FND) embedded in the transparent polymer.

In an embodiment, the fiducial marker composition comprises a marker complex comprising a fluorescent nanodiamond and a contrast agent for a nonfluorescent imaging method.

In an embodiment, the method of making a fiducial marker composition comprises immobilizing a fluorescent nanodiamond (FND) on a surface of a substrate, and coating the immobilized FND and surface with an inert top coat.

Methods of using FNDs as fiducial markers are also disclosed.

In an embodiment, an imaging method comprises contacting a sample with a fiducial marker composition disclosed herein; acquiring a plurality of fluorescent images of a target in the sample and a FND; and correcting target position in each image by aligning the position of the FND in all images.

In an embodiment, a super-resolution imaging correction method comprises determining position coordinates of each of m fluorescent nanodiamonds (FNDs) in each image of a plurality of n images by a Gaussian fitting of the point spread function of each FND in each image, wherein m≥4 and n>1; displacing each image to align the coordinates of a first FND (FND1) in all images; for each FND other than FND1, calculating the center of the distribution of positions of the FND over all n displaced images; and displacing each image such that the variance in position of all FND other than FND1 is minimized over all images.

These and other advantages, as well as additional inventive features, will be apparent from the following Drawings, Detailed Description, Examples, and Claims.

### BRIEF DESCRIPTION OF THE DRAWINGS

The following is a brief description of the drawings wherein like elements are numbered alike and which are presented for the purposes of illustrating the exemplary embodiments disclosed herein and not for the purposes of limiting the same.

FIG. 1 shows graphs of fluorescence intensity as a function of image Frame measured from nanogold or nanodiamond fiducial markers in fluorescent microscopy imaging.

FIG. 2 shows images of nanogold or nanodiamond fiducial markers and their associated expected and observed standard deviation of position errors in fluorescent microscopy imaging.

FIG. 3 is a histogram showing the observed standard deviation of position errors in fluorescent microscopy imaging along the X or Y axis for nanogold or nanodiamonds fiducial markers.

FIG. 4 is a multiplexed Super-Resolution view of the Immune Synapse obtained by sequential imaging of three T-Cell Receptor micro-complex forming proteins (LAT, SLP76, and pZeta) in a Jurkat T-Cell with simultaneous imaging of an Alexa Fluor-647 labeled-antibody against one of the three proteins and FNDs to eliminate drift during and between imaging of each sequentially imaged protein. Average localization precision=3.61 nm; Average alignment precision=2.07 nm.

FIG. 5 is a transmission electron micrograph of unstained fluorescent nanodiamonds (˜5 nm).

FIG. 6 compares three techniques (cross correlation, fiducial correction, and point correction) to correct stage movement in acquired SMLM images. Panels A-C show images of a FND after correction by cross correlation, fiducial correction, or point correction, respectively; panels D-F show 3-D histogram plots of the localization distribution of the images of panels A-C; panel G is a histogram showing the uncertainty ratio after correction by each of the methods; and panel H is a histogram of the X/Y localization ratio after correction by each of the methods.

FIG. 7A-F, panels A and B present plots showing the observed standard deviation as a function of the expected standard error of the mean for an FND (panel A) and an ALEXA FLUOR-647-labeled antibody (A647) (panel B), panels C and D present plots of the X-Y distribution of FND and the ALEXA FLUOR-647-labeled antibody, respectively; panels E and F show images (E) and 3-D histograms (F) of the visualization and localization of the antibody after the three types of correction.

FIG. 8A-E present schematic diagrams illustrating the steps of the point correction method using four FND fiducial markers in each image frame.

FIG. 9A-G present plots and images characterizing the distribution of multiple localizations from a single light-emitting source.

FIG. 10 presents images of the same FND using different magnification settings;

FIG. 11 is a schematic diagram illustrating multiplexed antibody size-limited dSTORM (madSTORM): an Alexa-647-conjugated antibody bound to the fixed cell sample and imaged using antibody size-limited dSTORM (FIG. 11A), they are unbound using a stripping buffer and their fluorescence is photobleached (FIG. 11B), then the cell sample is bound by a new Alexa-647-conjugated antibody, imaged, unbound, and photobleached (FIG. 11C, D).

### DETAILED DESCRIPTION

Fiducial marker compositions comprising fluorescent nanodiamonds (FNDs) and methods of making and using the fiducial marker compositions are disclosed.

FNDs are bright fluorescent probes that do not blink or bleach. Additionally, FNDs have broad fluorescence excitation and emission peaks, and the fluorescence intensity can be readily controlled by the size of the FND, the number of fluorescent centers produced in the nanodiamonds, or in situ through the application of a weak magnetic field (specifically for the case of NV−, or negative nitrogen vacancy centers) (Sarkar, S. K. et al. (2014) Biomed Opt Express 5(4): 1190-1202). These properties make FNDs ideal fiducial markers for fluorescence microscopy. The inventors have shown that FNDs outperform current fiducial markers for fluorescence microscopy in head-to-head comparisons, and offer a number of important advantages over current fiducial markers, such as gold nanoparticles or fluorescent beads.

In some embodiments, a fiducial marker composition is disclosed. In an embodiment, the fiducial marker composition comprises a substrate, and a fluorescent nanodiamond (FND) immobilized on a surface of the substrate. A variety of different immobilization techniques can be used. Depending on the immobilization technique, a top coat can be added to more permanently immobilize the FND. For example, the substrate and immobilized FND are at least partially top coated with an inert material such as silica (SiO₂). In another embodiment, the fiducial marker composition comprises a substrate, a transparent polymer immobilized on a surface of the substrate, and a fluorescent nanodiamond (FND) embedded in the transparent polymer, and optionally comprising an inert top coating. In another embodiment, the fiducial marker composition comprises a marker complex comprising a fluorescent nanodiamond and a contrast agent for a nonfluorescent imaging method.

The FND can be immobilized on the substrate with a polymer. The polymer can be a charged polymer or a transparent polymer. Examples of a charged polymer include polypeptides, both naturally occurring or synthetic, such as the homopolymers poly-L-lysine and poly-L-arginine. Examples of a transparent polymer include siloxanes such as poly(dimethylsiloxane) (PDMS), poly(meth)acrylates such as poly(methyl acrylate) and poly(methyl methacrylate), polycarbonates, polyphosphonates, poly(vinyl butyral), polyesters, and polyimides. Alternatively, the FNDs can be dispersed in gels such as agarose or polyacrylamide gels. The FNDs can be suspended in a solution or melt of the polymer at a suitable concentration and then the suspension or melt can be dispersed on the substrate by any method known in the art, for example by pipetting or by spin-coating. Alternatively, the substrate can first be coated with the polymer, and then subsequently the FNDs, in the form of a suspension, e.g., can be dispersed onto the polymer-coated substrate. The polymer coating can be patterned before or after dispersing the fluorescent nanodiamond onto the substrate. The substrate can also first be patterned with the polymer, and then subsequently a suspension of the FNDs can be dispersed onto the polymer pattern on the substrate. Any conventional patterning techniques can be used to generate the polymer pattern, for example photolithography or soft lithography. The FND can also be immobilized on the surface of the substrate by functionalizing the surface of the substrate with a functional group that reacts with the FND or with a functional group of a functionalized FND, and applying a solution of FND or functionalized FND to the functionalized surface. The functionalized substrate surface can optionally be patterned. In any of the methods of immobilizing FNDs to the surface, the nonimmobilized FNDs can be removed by washing the surface with a suitable solution, such as water or a buffer.

Any suitable methods known in the art for surface functionalization of the substrate can be used. One method of covalently derivatizing a silica or glass surface is silanation with an organofunctional tri(C₁₋₈alkoxy)silane or trichlorosilane, for example amino(C₁₋₈alkyl)tri(C₁₋₈alkoxy)silanes, amino(C₁₋₈alkyl)trichlorosilanes, mercapto(C₁₋₈alkyl)tri(C₁₋₈alkoxy)silanes, hydroxy(C₁₋₈alkyl)tri(C₁₋₈alkoxy)silanes, hydroxy(C₁₋₈alkyl)trichlorosilanes, carboxy(C₁₋₈alkyl)tri(C₁₋₈alkoxy)silanes, epoxy(C₁₋₈alkyl)tri(C₁₋₈alkoxy)silanes, N-(amino C₁₋₈alkyl)(amino C₁₋₈alkyl)tri(C₁₋₈alkoxy)silanes, and the like. Specific examples include 3-aminopropyltriethoxysilane (APTES), (3-aminopropyl)-dimethylethoxysilane (APDMES), N-(2-aminoethyl)-3-aminopropyltrimethoxysilane (AEAPS), 3-aldehydepropyltrimethoxysilane (APMS), mercaptopropyltrimethoxysilane (MPTMS), and mercaptopropyltriethoxysilane (MPTES), and others, such as aminotriethoxysilane. Other specific examples of derivatizing agents particularly suited for modifying the physical characteristics (e.g., hydrophilicity) of a silica surface include 2-[methoxy(polyethyleneoxy)propyl]trimethoxysilane, 2-[methoxy(polyethyleneoxy-propylenoxy)propyl] trimethoxysilane, (C₁₋₃₂alkyl)trichlorosilanes such as octadecyltrichlorosilane.

Where the derivatization agent includes a functional group, the functional group can be further derivatized. Thus, it is also possible to use a functionalized trialkoxysilane or trichlorosilane as a linking group between the silica surface and another molecule, such as a monomer or hydrophilic polymer (e.g., methyl cellulose, poly(vinyl alcohol), dextran, starch, or glucose). The functional group of the trialkoxysilane or trichlorosilane is selected to react with the other molecule, and can be any of those described above, for example, a vinyl, allyl, epoxy, acryloyl, methacryloyl, sulfhydryl, amino, hydroxy, or the like. The functionalization can be simultaneous or stepwise.

Noncovalent functionalization of silica surfaces can be based on electrostatic interactions due to the negative nature of silica above about pH 3.5. For example, positively charged polymers can adsorb electrostatically to the silica surface.

Any suitable methods known in the art for surface functionalization of the FND can be used. One method of functionalizing the FND is to encapsulate the FND with a silica as described in WO2014014970; or in Bumb, A. et al. (2013) Journal of the American Chemical Society 135(21): 7815-7818. Functionalized silica precursors can be used in the encapsulation process to obtain a functionalized silica coating. A silica-coated FND can also be derivatized by reaction with a reagent, such as the cross linker N-Hydroxysulfosuccinimide (NHS) sodium or a derivatized NHS, with the FND and a silane such as an alkoxysilane. (WO2014014970; Bumb et al. 2013) FNDs can be oxidized by acid treatment, producing anionic carboxylate groups on the nanodiamond surface (Chang, B. M. et al. (2013) Advanced Functional Materials 23(46): 5737-5745.). Oxidized FNDs adsorb various biomolecules with positively charged groups, such as proteins with amino groups (Ermakova A. et al. (2013) Nanoletters 13:3305-3309) or poly lysine (Fu, C.-C. et al. (2007) Proc Natl Acad Sci USA 104(3):727-732). Such oxidized FNDs can be further functionalized with amino groups. For example the surface carboxylate groups of oxidized FNDs can be reacted with reagents such as N-(3-dimethylaminopropyl)-N-ethyl-carbodiimide hydrochloride (Fu et al. 2007). FNDs have also been pegylated and further derivatized (Chang et al. 2013).

Alternatively, the transparent polymer to be immobilized on the substrate can be first formed (e.g., cast) as a sheet or other shape prior to immobilization of the shape on the substrate. FNDs can be mixed in the solution of the transparent polymer such that upon forming the solution into a shape, the FNDs are located at random positions throughout the shape, resulting in the FNDs being in different focal planes within the transparent shape. Alternatively, after immobilization of the shape on the surface, FNDs can be dispersed and immobilized on the surface of the shapes. In an embodiment, a transparent polymer is cast into a sheet, which is then divided (e.g., cut) into smaller shapes. If the immobilized transparent polymer shapes vary in height, the FNDs immobilized on the surfaces of the transparent polymer shapes will have FND fiducial markers in multiple focal planes and therefore can be used to provide superior correction of 3-dimensional imaging methods.

The density of FNDs on the substrate can be between about 10 to about 500 FND per 100 μm², specifically about 10 to about 300 FND per 100 μm², more specifically about 10 to about 50 FND per 100 μm², about 50 to about 150 FND per 100 μm², or about 150 to about 300 FND per 100 μm².

In any of the embodiments, at least two FNDs can be immobilized in the transparent polymer or immobilized on the surface of the substrate such that the distance between the FND and the substrate surface is not identical for the two FNDs.

The inert top coating can be an inert material such as a silica, alumina, or a hybrid organic-inorganic material such as alucone. The top coating can be made by any method known in the art. For example, a silica or alumina top coating can be made by sputter-coating the composition with silica or alumina, respectively. The inert top coating on the compositions eliminates the possibility of any FND motion, isolates the composition from any sample, and permits reuse of the composition.

As used herein, the term “nanodiamond” refers to a nanodimensioned diamond particle. “Diamond” as used herein includes both natural and synthetic diamonds from a variety of synthetic processes, as well as “diamond-like carbon” (DLC) in particulate form. The diamond can be of any shape, e.g., rectangular, spherical, cylindrical, cubic, or irregular, provided that at least one dimension is nanosized, i.e., less than: about 1 micrometer, about 800 nm, about 500 nm, about 250 nm, about 200 nm, about 150 nm, about 100 nm, about 90 nm, about 80 nm, about 70 nm, about 60 nm, about 50 nm, about 40 nm, about 30 nm, about 20 nm, about 20 nm, or about 10 nm. Specifically, the largest dimension of a nanodiamond should be less than the diffraction limited spot size of the microscope defined by the Abbe diffraction limit at the imaging conditions.

As is known in the art, accurate determination of particle dimensions in the nanometer range can be difficult. In an embodiment, the dimension of the nanodiamonds is determined using their hydrodynamic diameter. The hydrodynamic diameter of the nanodiamond or an aggregate of nanodiamonds can be measured in a suitable solvent system, such as an aqueous solution. The hydrodynamic diameter can be measured by sedimentation, dynamic light scattering, or other methods known in the art. In an embodiment, hydrodynamic diameter is determined by differential centrifugal sedimentation. Differential centrifugal sedimentation can be performed, for example, in a disc centrifuge. In an embodiment, the hydrodynamic diameter is a Z-average diameter determined by dynamic light scattering. The Z-average diameter is the mean intensity diameter derived from a cumulants analysis of the measured correlation curve, in which a single particle size is assumed and a single exponential fit is applied to the autocorrelation function. The Z-average diameter can be determined by dynamic light scattering with the sample dispersed in, for example, deionized water. An example of a suitable instrument for determining particle size and/or the polydispersity index by dynamic light scattering is a Malvern Zetasizer Nano.

As used herein, the term “fluorescent nanodiamond” (abbreviated as “FND”) refers to nanodiamonds that exhibit fluorescence when exposed to an appropriate absorption (excitation) spectrum. Fluorescent nanodiamonds are commercially available from a number of sources, e.g. Adámas Nanotechnologies (Raleigh, N.C.) or Sigma-Aldrich. The size of the FND can be about 5 nm to about 200 nm.

The fluorescence of nanodiamond particles is based on color centers incorporated into the diamond lattice. This fluorescence can be caused by the presence of nitrogen-vacancy (NV) centers, where a nitrogen atom is located next to a vacancy in the nanodiamond, which provide red fluorescence, and/or nitrogen-vacancy-nitrogen (N—V—N or H3) centers, which emit green light. The optical properties of the NV center are well suited for bioimaging applications, with optical excitation from 490-560 nm and emission in the red\near infrared (637-800 nm) away from most autofluorescent cell components. The emission also occurs in a spectral window of low absorption attractive for biological labeling due to greater penetration of light in the surrounding tissue. The intensity of the luminescence emitted from nanodiamonds containing NV centers depends on the number of NV centers in a particle. The N—V-N center emits green fluorescence with a maximum around 530 nm when excited by blue light. Numerous color centers, other than NV and N—V-N centers, have been fabricated and characterized in nanodiamonds. Examples of other color centers fabricated in FNDs include a chromium (Cr) center, a silicon vacancy (Si—V) center, and Nickel (Ni)-nitrogen complexes emitting at 797 nm (Aharonovich, I. et al. Phys. Rev. B 81, 121201, 15 Mar. 2010; Vlasov I. I. et al. Adv. Mater. 2009, 21, 808-812; Rabeau J. R. et al. Appl. Phys. Lett. (2005)86, 131926). A nanodiamond produced with any suitable color center(s) can be used in the compositions and methods disclosed herein. Thus in the disclosed compositions and methods, the FND can be a multicolor FND with at least two color centers. For example, a multicolor FND can include both an NV and N—V-N centers or a multicolor FND can include N—V—N and Si—V centers. One advantageous feature of color centers within a diamond is that they do not photobleach or blink even under continuous high energy excitation conditions making them superior to conventional chromophores due to their unprecedented photostability. Furthermore, since color centers are embedded within the diamond matrix their fluorescence properties are not affected by surface modification or environmental conditions such as solvent, pH, and temperature.

“Substrate” refers to a material or group of materials having a rigid or semi-rigid surface or surfaces. Examples of such materials include polymers (e.g., polycarbonate, polyolefin, polyethylene terephthalate, poly(meth)acrylates), glass, and silicon wafers, specifically glass, more specifically quartz. In some aspects, at least one surface of the substrate is substantially flat, although in some aspects it may be desirable to have, for example, wells, raised regions, pins, etched trenches, or the like. In certain aspects, the substrate can take the form of beads (e.g., latex beads), gels, microspheres, or other geometric configurations.

In another aspect, methods of making a fiducial marker composition are disclosed.

In an embodiment, the method comprises immobilizing a fluorescent nanodiamond (FND) on a surface of a substrate, and coating the immobilized FND and surface with an inert top coating such as SiO₂. The immobilized FND and the substrate surface can be coated by any suitable method, for example sputter-coating the substrate surface. The inert top coating can have a thickness of about 50 nm to about 300 nm, specifically, about 100 nm to about 200 nm, more specifically, about 150 nm.

The FND can be immobilized on the substrate surface by any known method, e.g., any of the methods disclosed herein. The FND can be immobilized by applying a mixture of the FND in an aqueous solution of polymer to the surface of the substrate. The FND can also be immobilized by coating the surface with a polymer; and dispersing FND onto the polymer coating. Optionally, the polymer coating can be patterned before or after the FND is dispersed onto the coating. The polymer can be e.g., a transparent polymer or a charged polymer, such as polypeptides, for example poly-L-lysine or poly-L-arginine. Examples of suitable charged polymers and transparent polymers were disclosed above. The FND can also be immobilized on the substrate by immobilizing a pre-cast object comprising transparent polymer on the substrate surface, wherein an FND is contained within the object or on a surface of the object. The FND can also be immobilized by functionalizing the surface of the substrate with a functional group that reacts with the FND or a functionalized FND; and applying a solution of FND or functionalized FND to the functionalized surface. In any of the embodiments, FNDs that are not immobilized can be removed by washing the substrate surface with a suitable solution, such as water or a buffer.

In another aspect, an imaging method is disclosed.

In an embodiment, the method comprises contacting a sample with a fiducial marker composition disclosed herein; acquiring a plurality of fluorescent images of a target in the sample and a FND; and correcting target position in each image of the plurality of images for drift and alignment by registering each image with the position of the fluorescence of the FND.

The imaging method can be a multi-modal imaging method in which at least one additional imaging technique is used that differs from fluorescence imaging. The additional imaging method can be magnetic resonance imaging (MRI), computerized tomography (CT) imaging, X-ray imaging, or electron microscopy. In some embodiments of such a multi-modal imaging method, the FND is encapsulated in a liposome, and the liposome further encapsulates a contrast or imaging agent for the additional imaging technique. In some embodiments of such a multi-modal imaging method, the FND is coupled to the contrast or imaging agent for the additional imaging technique. Examples of the contrast or imaging agent include an osmium-containing moiety, a gadolinium containing moiety, a dysprosium containing moiety, or a high electron density (Z) material. An example of an osmium-containing moiety is osmium tetroxide. Examples of gadolinium containing moieties include gadolinium chelates such as gadolinium-diethylenetriaminepentaacetic acid dimeglumine ([NMG]2Gd-DTPA]), OMNISCAN™ (Gd diethylenetriaminepentaacetic acid bis(methylamide)), PROHANCE™ (Gd(10-(2′-hydroxypropyl)-1,4,7,10-tetraazacyclododecane-N,N′,N″-triacetic acid)), and others disclosed in WO1996010359, as well as polyaminopolycarboxylic acid complexes of gadolinium. Examples of dysprosium-containing moieties include dysprosium (Dy) chelates such as Dy-diethylenetriaminepentaacetic acid bis(methylamide) and others disclosed in WO1996010359. Examples of a high Z material include gold, uranium, or tungsten.

Contacting a sample with a disclosed fiducial marker composition can be performed by a variety of methods. Methods of contacting the sample with the fiducial marker composition include pipetting or embedding the sample onto the fiducial marker composition, or the fiducial marker composition onto the sample; injecting a fiducial marker composition into a sample; or feeding a fiducial marker composition to an organism. A fluorescent nanodiamond can bind to a sample via a functional group or ligand on the FND surface.

A “sample” refers to a specimen containing a target to be imaged. A sample can be a solution, a suspension, a cell, a tissue, an organ, a cellular membrane, an organelle, or an organism.

The term “target” refers to a molecule or molecular complex of interest that is to be imaged. Targets may be naturally-occurring or man-made molecules. Also, they can be employed in their unaltered state or as aggregates with other species. Examples of targets include biomolecular complexes (e.g., a T cell receptor microcluster), proteins (e.g., cell membrane receptors, or antibodies), drugs, oligonucleotides, nucleic acids, peptides, cofactors, lectins, sugars, polysaccharides, cells, cellular membranes, and organelles.

In an embodiment, a super-resolution imaging correction method comprises determining position coordinates of each of m fluorescent nanodiamonds (FNDs) in each image of a plurality of n images by a Gaussian fitting of the point spread function of each FND in each image, wherein m≥4 and n>1; displacing each image to align the coordinates of a first FND (FND1) in all images; for each FND other than FND1, calculating the center of the distribution of positions of the FND over all n displaced images; and displacing each image such that the variance in position of all FND other than FND1 is minimized over all images. In an embodiment, FND1 is selected to be the FND with the greatest intensity.

The imaging method to be corrected can be any imaging method in which FNDs are suitably used as fiducial markers. Examples of such imaging methods include fluorescence microscopy, electron microscopy, MRI, CT, and X-ray imaging, specifically any super-resolution microscopy methods, such as single molecule localization microscopy (SMLM) methods which include photo-activation localization microscopy (PALM), stochastic optical reconstruction microscopy (STORM), and direct STORM (dSTORM). The imaging method can be a two-dimensional (2-D) or three-dimensional (3-D) imaging method. Further, the imaging method can be multi-modal.

Methods to determine position or position coordinates of an object in an image obtained by the particular imaging method are well known in the art, and any suitable methods can be used. Software to determine position in an image is available, both commercially and from various free internet sources. For example, several free plug-ins for Image-J or FIJI such as Mosaic, Track Mate, multi tracker, and Thunderstorm. In addition position determination can be performed in proprietary software from Nikon (e.g., NIS-A N-STORM) or in the MatLab or LabView environments. See for example (Chenouard, N. et al. (2014) Nat Meth 11(3): 281-289.) for a compendium of recent tracking software. Several algorithms can be used for position determination including 2-Dimensional Gaussian fitting of the fluorescence intensity distribution, centroid determination, and local maximum fitting.

The displacement can be at least one of a translation, a rotation, or a dilation/contraction.

Displacing each image such that the variance in position of all FND other than FND1 is minimized can be performed by any suitable method. In an embodiment, displacing each image such that the variance in position of all FND other than FND1 is minimized comprises calculating the mean of the center of the distribution of positions of the FND over all n displaced images for all FND other than FND1; calculating the mean position of all FND other than FND1 in each image; and displacing a given image to minimize the difference between the mean of the center of the distribution of positions of the FND over all n displaced images for all FND other than FND1 and the mean position of all FND other than FND1 in the given image. An alternative approach involves determining the positions of the m fiducial markers in one reference image. Each subsequent image is transformed to minimize the sum of the squares of the differences between the positions of the fiducial markers in the reference and transformed image. The differences can be weighed by the brightness of each fiducial marker to increase the robustness of the transformation process. The transformation can be a simple rigid body translation, a combination of a rigid body translation and a rotation, a combination of a rigid body translation, a rotation, and a uniform dilation or contraction, or a non-linear mapping of the transformed image onto the reference image. The following examples are merely illustrative of the fiducial marker compositions and methods disclosed herein, and are not intended to limit the scope hereof.

### EXAMPLES

### Example 1. Comparison of FNDs and Gold Particles in Fluorescent Imaging

Fluorescent nanodiamonds fiducial marker slides were generated by spin coating FNDs onto a glass slide with polylysine.

The FND fiducial markers were compared to commercially available nanogold particle fiducial marker slides.

Total internal reflection fluorescence (TIRF) confocal images were acquired using a NIKON ECLIPSE Ti inverted microscope. The fluorophore ALEXA FLUOR® 647, fluorescent nanodiamonds (FNDs), and gold fiducial markers were excited by a 647 nm acousto-optic tunable filter (AOTF)-modulated NIKON LU-NB solid state laser (125 mW). Emission was collected by a Nikon 100x SR Apochromat TIRF objective lens (1.49 NA) and imaged with an Andor iXon Ultra 897 EMCCD camera (512×512, 16 μm square pixels). Direct stochastic optical reconstruction microscopy (dSTORM) localization of TIRF confocal images was performed using Thunderstorm plugin (ver. 1.2) in Image J. Point correction of dSTORM localization data based on fluorescence from FNDs was performed using customized code written in MATLAB. Unless otherwise stated images were acquired with an integration time of 200 ms.

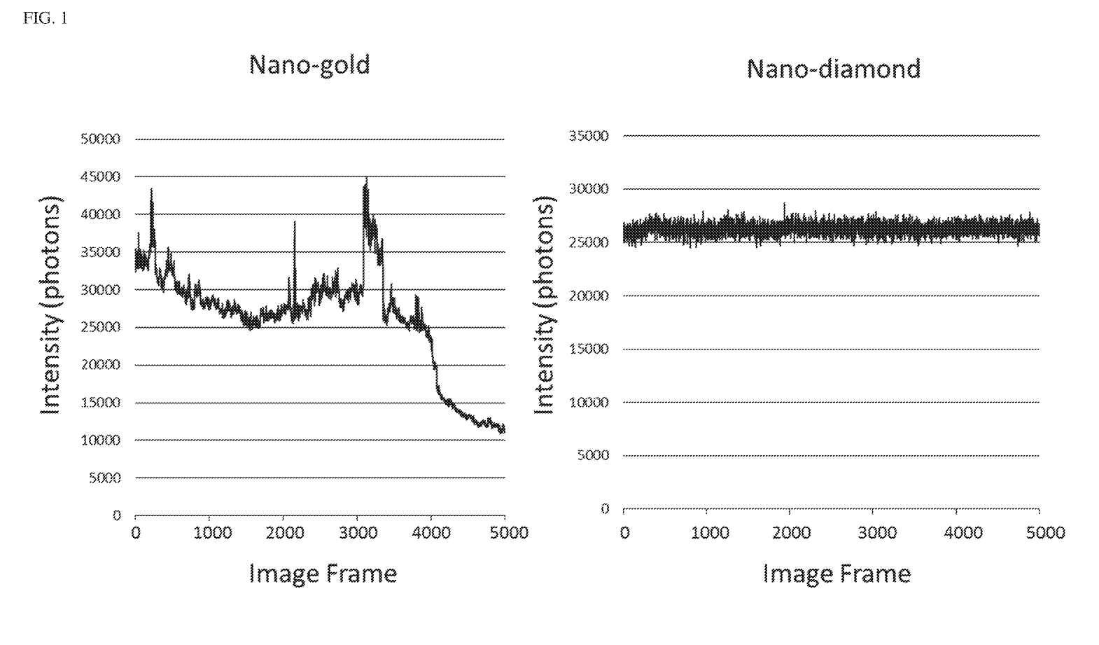

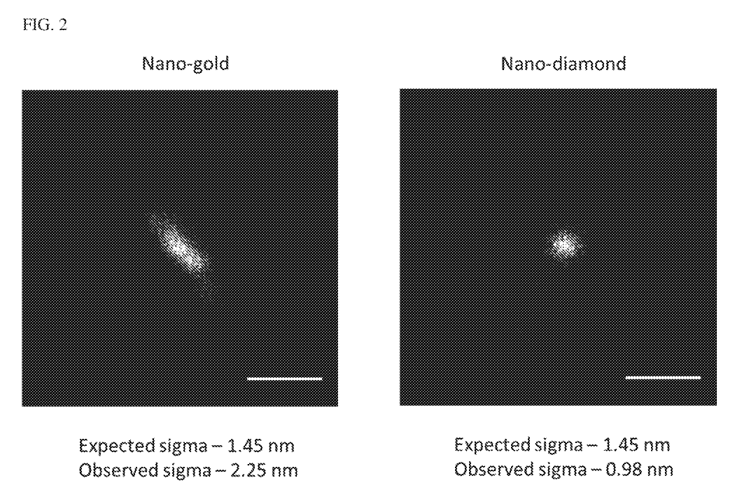

FIG. 1 shows the intensity over time measured from each type of fiducial marker. The FND marker displays better temporal stability than the nanogold marker.

FIG. 2 shows an image of a nano-gold and a nanodiamond marker, respectively, and values for the expected and observed standard deviation (sigma) of the position error for each. The position error for the FND fiducial markers has an observed standard deviation that is less than half that of the nanogold fiducial markers.

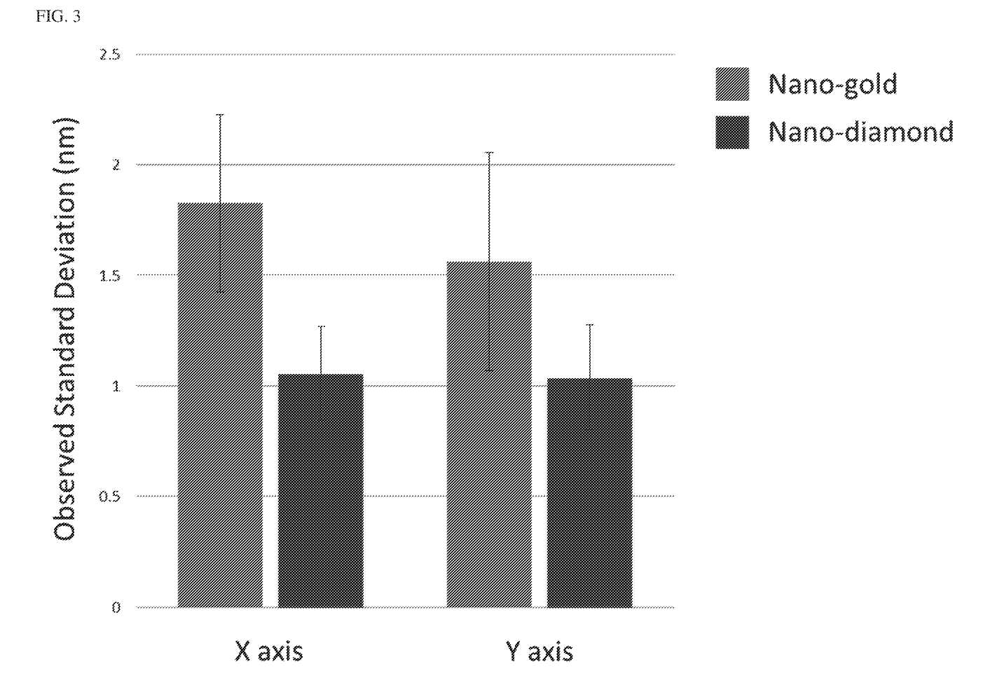

FIG. 3 presents a histogram comparing the observed standard deviation of lateral resolution of nanogold or nanodiamonds fiducial markers along the X axis and Y axis, respectively. The observed standard deviation is smaller in each direction for the FND fiducial markers than for the nanogold fiducial markers.

Thus FND fiducial markers afford higher accuracy position tracking and better stability compared to the nanogold fiducial markers.

### Example 2. FND Fiducial Markers in Transmission Electron Microscopy

FND fiducial markers were tested for their utility in imaging via Transmission electron microscopy (EM).

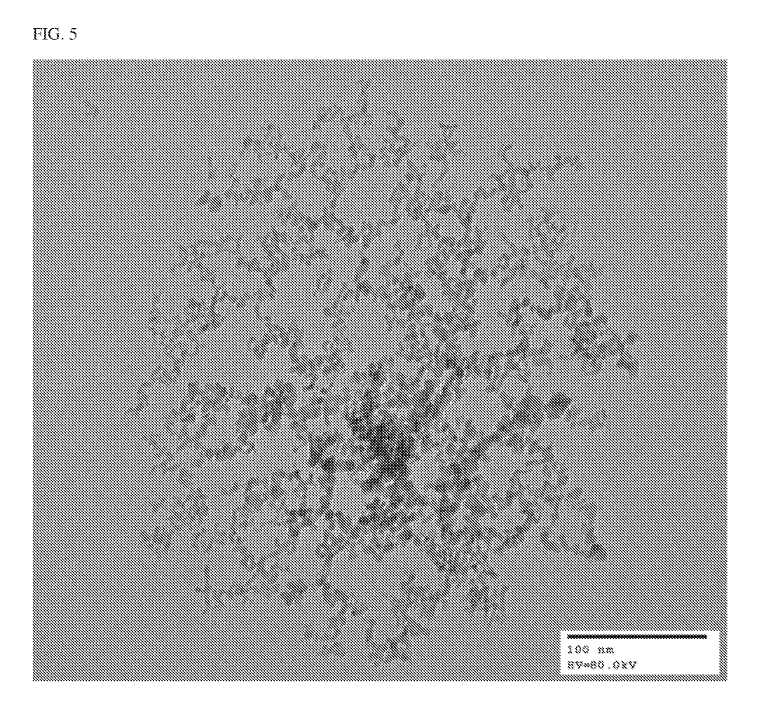

FNDs having a particle size of about 5 nm were spread and imaged via TEM without staining. FIG. 5 shows an electron micrograph of such an FND sample, showing that ˜5 nm FNDs provide good contrast in TEM without staining.

### Example 3. Multiplexed dSTORM

In this example we describe a new algorithm using fluorescent nano-diamond (FND) fiducial markers to register samples for drift correction and alignment, coupled with a novel imaging technique that potentially allows unlimited multiplexing of fluorescent probes using dSTORM. Using the FND-based drift correction and multiplexed dSTORM, we probe the nano-scale topography of molecular components of the T cell receptor (TCR) microcluster and other cellular structures near the activated membrane surface of a T cell.

The resolution limit of light microscopy has been surpassed by techniques known collectively as super-resolution microscopy. However, there exists a spectral limit to light microscopy. Multicolor imaging is restricted to six colors due to the limited availability of non-overlapping wavelength profiles for different fluorescent probes. We have developed a novel technique to allow potentially unlimited multiplexed super-resolution imaging using direct stochastic optical reconstruction microscopy (dSTORM). In addition, we have developed a more precise method of registering samples for drift correction and alignment using fluorescent nanodiamond fiducial markers, achieving >2 fold improvement in precision over previous studies. Using this dSTORM technique, we have successfully visualized 20 different molecules in the same cell with an average localization precision of 2.5 nm and alignment precision of 3.5 nm. Simultaneously probing the spatial distribution of molecules involved in the TCR signaling cascade and other molecular networks will soon be possible with multiplexed dSTORM.

Engagement of the TCR leads to the formation of TCR microclusters that function as a basic signaling unit during T cell activation (Bunnell, S. C. et al. (2002) J Cell Biol 158(7): 1263-1275; Campi, G. et al. (2005) J Exp Med 202(8): 1031-1036). Moreover, differential transport and accumulation of these microclusters at the activated T cell surface leads to a structure called the immune synapse. While TCR microclusters have been studied extensively using conventional light microscopes, their nanostructure and the relative distribution of TCR signaling molecules are not well characterized due to the diffraction and spectral limits of light microscopy.

We have shown using PALM that molecular components of the TCR microcluster show distinct patterns of localization (Sherman, E. et al. (2011) Immunity 35(5): 705-720). However, imaging the relative distribution of TCR microcluster components using PALM has been hindered by the ability to image in only 2 colors. Moreover, we and others have observed artificial clustering of fluorescently-tagged molecules, in particular those tagged with the fluorescent protein PA-mCherry, leading us to seek new imaging modalities in visualizing the immune synapse (Wang, S. Y. et al. (2014) Proceedings of the National Academy of Sciences of the United States of America 111(23): 8452-8457).

### Point Correction

To compensate for stage movement during dSTORM acquisition, current correction methods either estimate the trajectory of stage drift by sampling localized peaks from the entire field of view (‘cross correlation’) or regression-based smoothing of localized positions from known fiducial markers (‘fiducial correction’). These methods require optimization of sampling/regression parameters for each image and, as shown below, do not adequately correct all stage movement. Rather than correcting stage movement post-image acquisition, a few studies have utilized hardware-based strategies to actively stabilize the stage in real time. However, they require complicated hardware and software modification, making them difficult to implement.

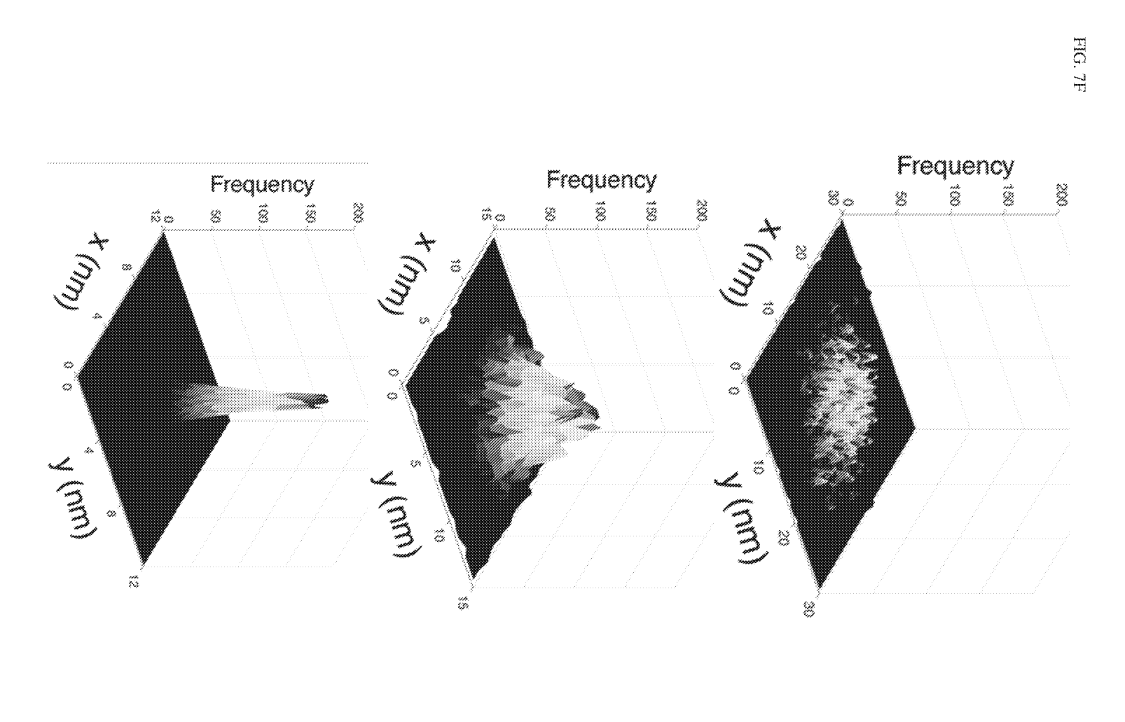

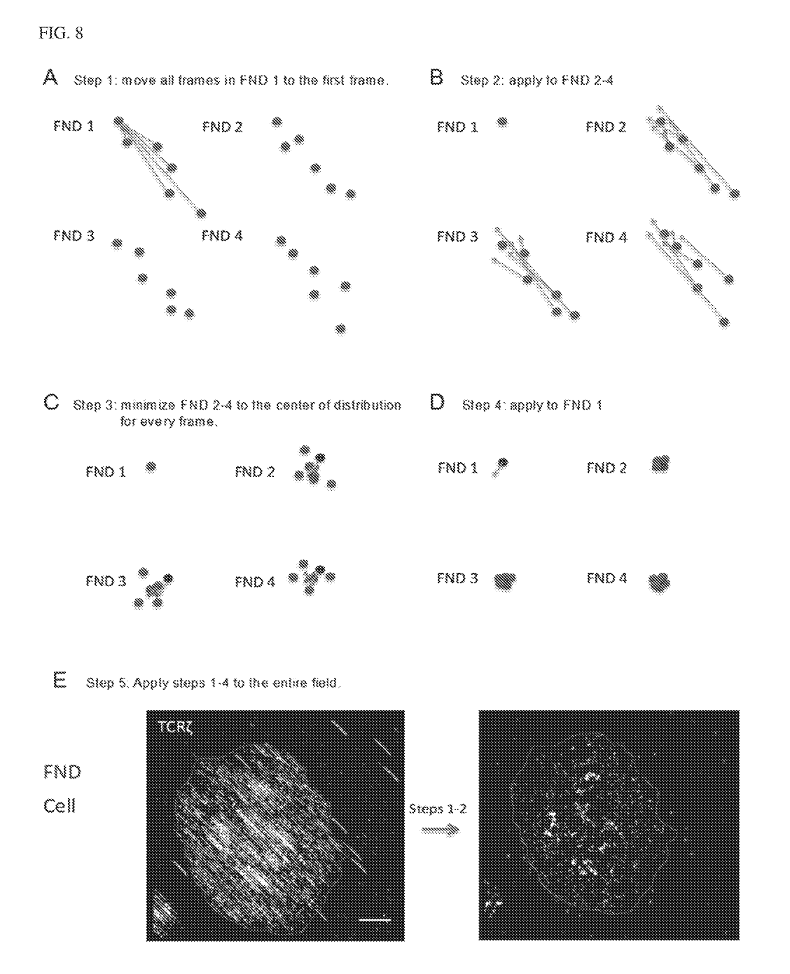

We have developed a new correction method called ‘point correction’ using FNDs as fiducial markers. FNDs are ideal SMLM fiducial markers since they are small (<100 nm), bright, photo-stable, and display a broad spectral range of fluorescence. Point correction is a 5 step process and requires >4 FND fiducial markers to be present in all image frames. First, the Gaussian peaks are localized for all FNDs, the brightest FND is designated as FND 1, and the X,Y positions of all localizations in FND 1 are moved to its position in the first frame (step 1; FIG. 8A). Next, the displacement from step 1 is applied to all other FNDs (step 2; FIG. 8B). Third, the center of localization distribution is defined for all FNDs other than FND 1, and the minimum distance between their localizations and centers of distribution is calculated as a single displacement for each frame (step 3; FIG. 8C). This displacement is then applied to FND 1 (step 4; FIG. 8D). Lastly, the displacements from steps 1 and 3 are applied to the entire image stack to correct stage movement (step 5; FIG. 8E).

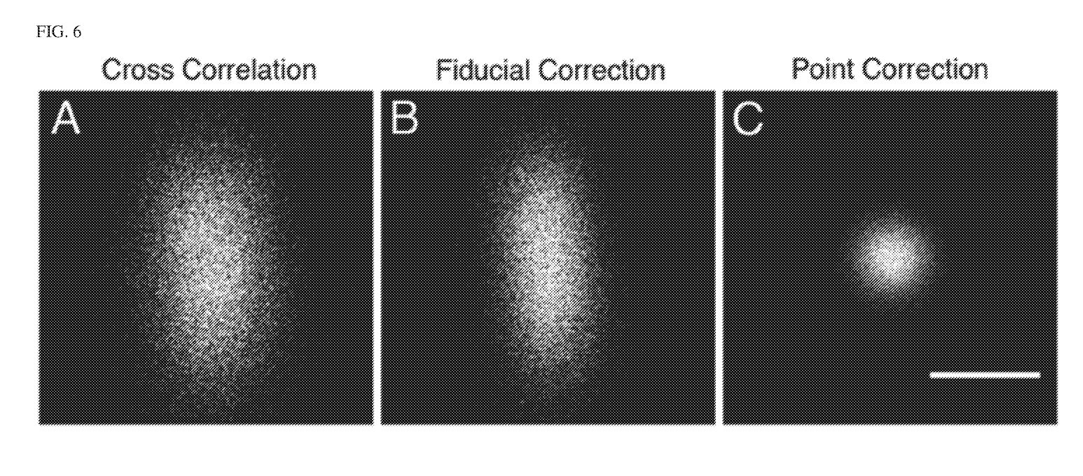

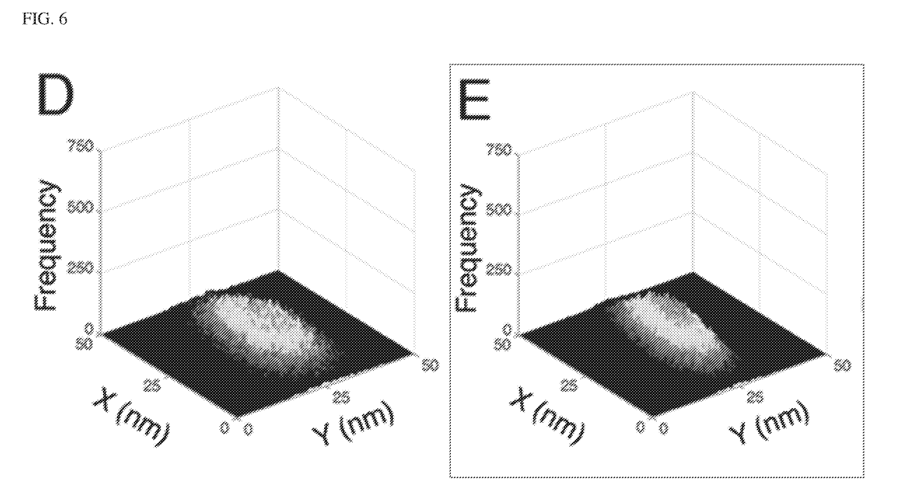

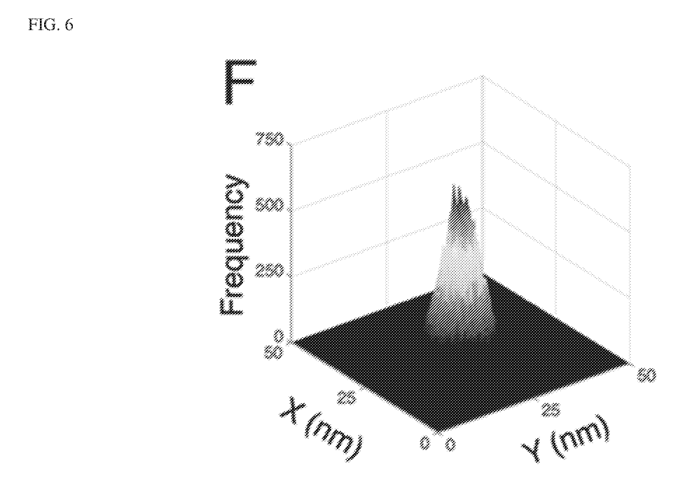

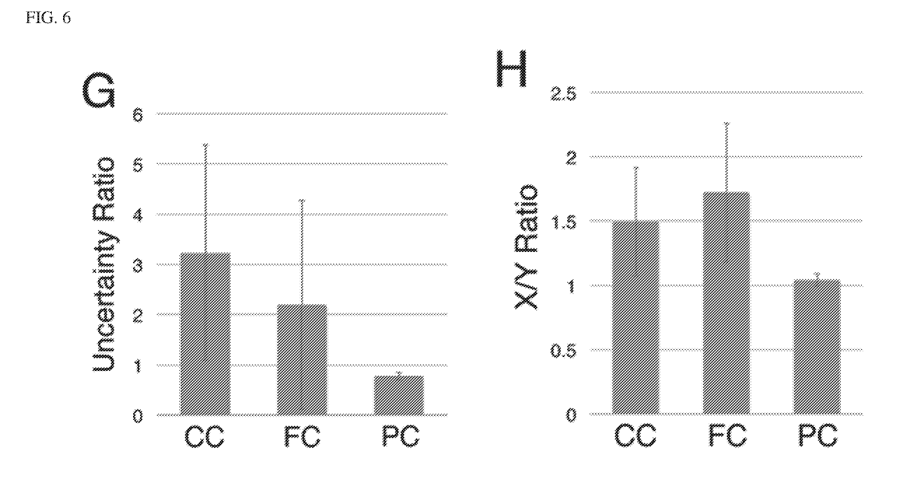

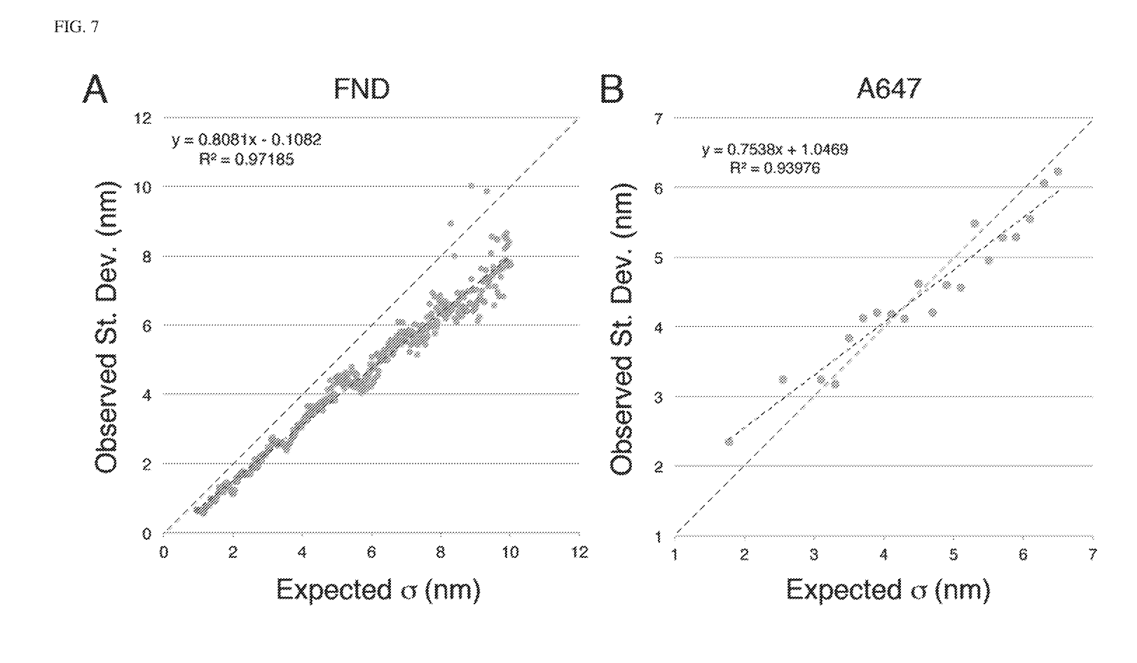

Using FNDs embedded on a Poly-L-Lysine-coated coverslip, we tested the ability of cross correlation, fiducial correction, and point correction to correct stage movement in the acquired SMLM images. While cross correlation and fiducial correction could compensate for large stage drift, they could not correct small stage vibrations, resulting in an elongated, non-symmetric distribution of localizations in the direction of the vibrations. (n=30,000 frames; FIG. 6, panels A-B and H). In contrast, the same FND corrected with point correction consistently yielded a symmetric distribution of localizations (FIGS. 6C and 6H). Also, the range of localization distribution was smaller with point correction compared to other correction methods, as visually evident from the 3D histogram plots (FIG. 6D-F) and by their standard deviation (SD) (6.0 nm, 11.3 nm [X,Y axis], cross correlation; 4.8 nm, 10.87 nm, fiducial correction; 3.8 nm, 3.9 nm, point correction). The SD of multiple localizations after point correction closely matched the average standard error of the mean (SEM; denoted by a) calculated for each localization (compare 3.8, 3.9 nm SD to 4.0 nm SEM, FIG. 6C, F) suggesting that all stage movement was corrected. In fact, repeated experiments showed that the observed SD of FND localizations was more precise than the predicted SEM, as evidenced by the low uncertainty ratio (calculated by SD/SEM) after application of point correction (0.71+0.07, PC; n=26 FNDs; FIG. 6G), whereas cross correlation and fiducial correction yielded 2-3 fold lower precision than expected (3.23+2.14, CC; 2.20+2.07, FD; n=26 FNDs; FIG. 6G).

### Distribution of Multiple Localizations

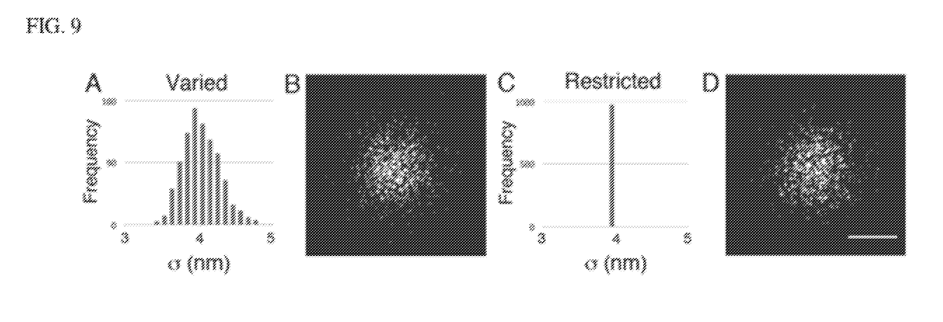

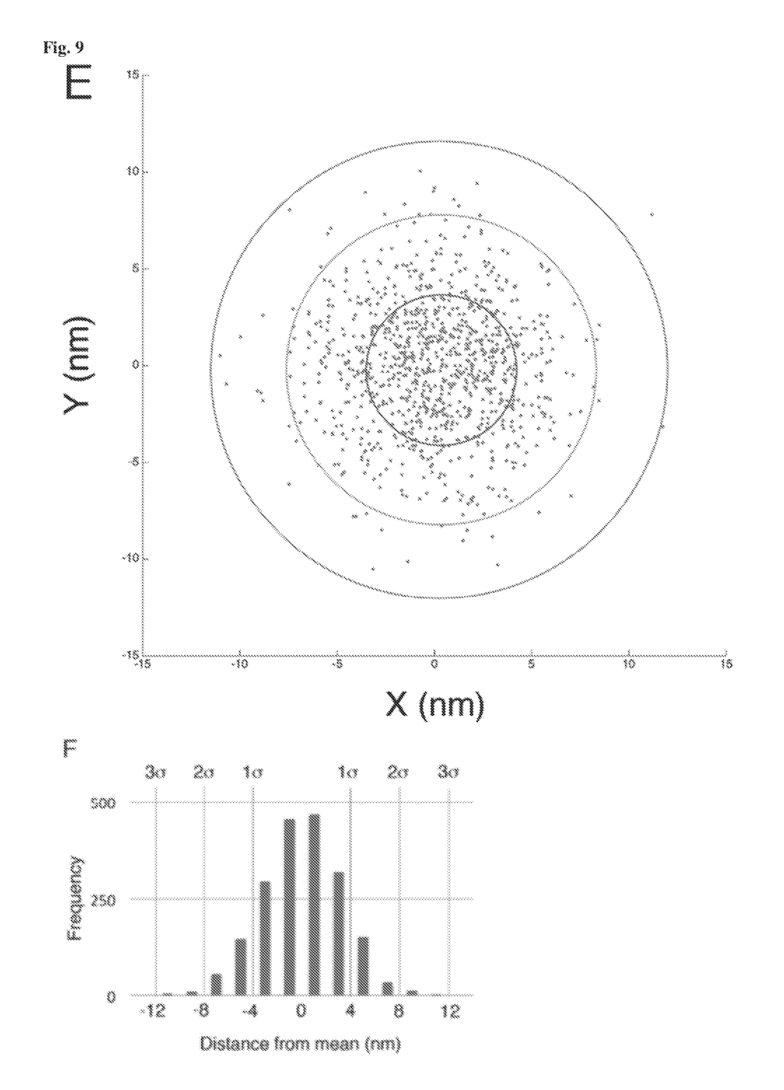

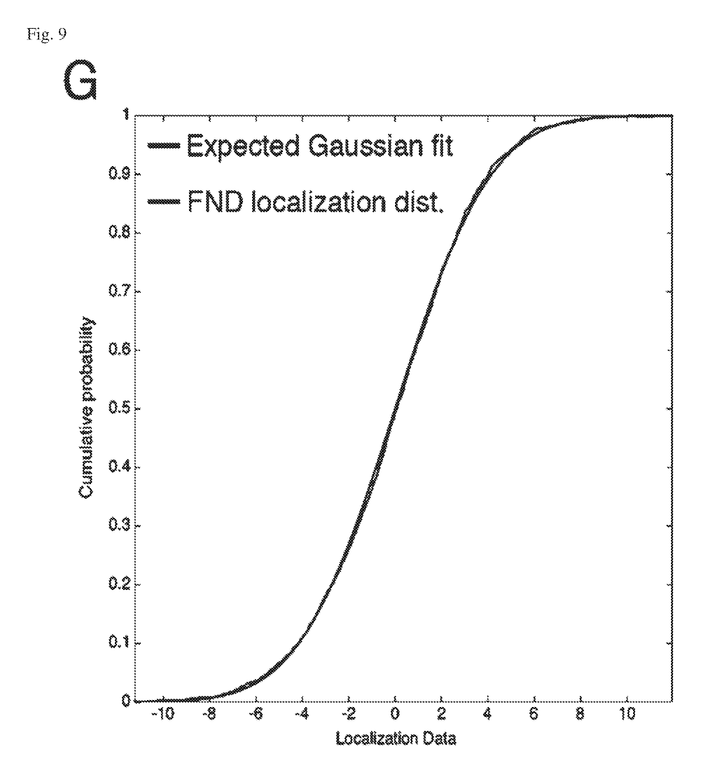

Next we sought to characterize the distribution of multiple localizations from a single light-emitting source. A previous study suggested that such a distribution reflected the stochastic variation in localization precision due to random fluctuations in fluorescence intensity during image acquisition. To test this we analyzed the localization distribution from a point correction-applied FND with a stochastic variation in uncertainty values (Varied; n=997 localizations; FIG. 9A; mean σ=4.0 nm) or restricted to a precision value of 4±0.01 nm (Restricted; n=997 localizations; FIG. 9C). We observed almost no difference in their respective SD values (3.3 nm, 3.5 nm, Varied vs. 3.3 nm, 3.4 nm, Restricted [X,Y axis]) or their net range of distribution (22.7 nm, Varied vs. 24.3 nm Restricted; FIG. 9B, D), showing that variation in precision is not a major component of the localization distribution.

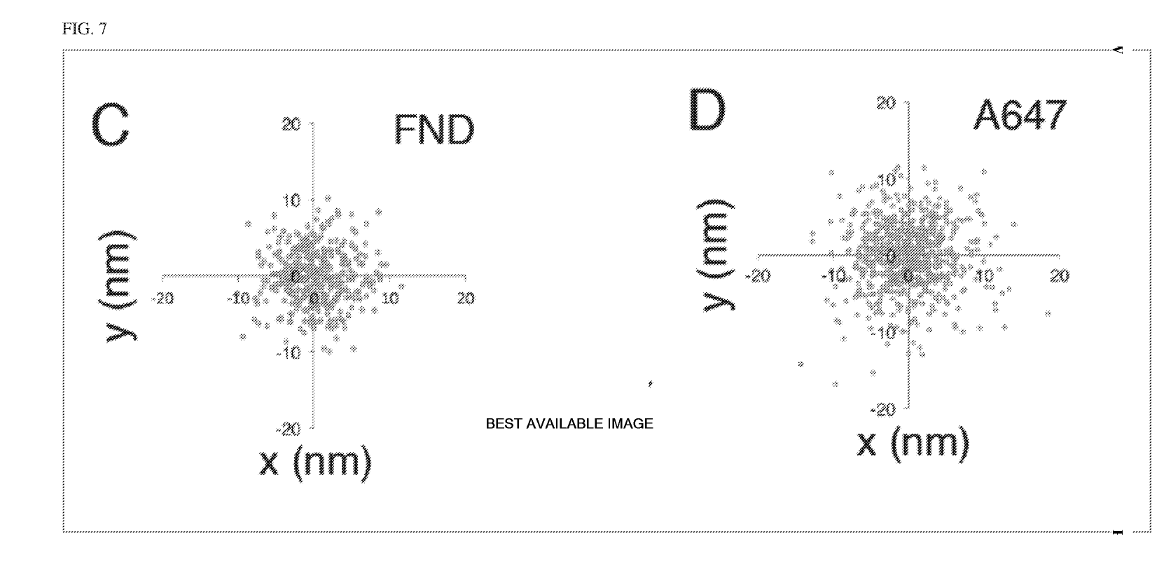

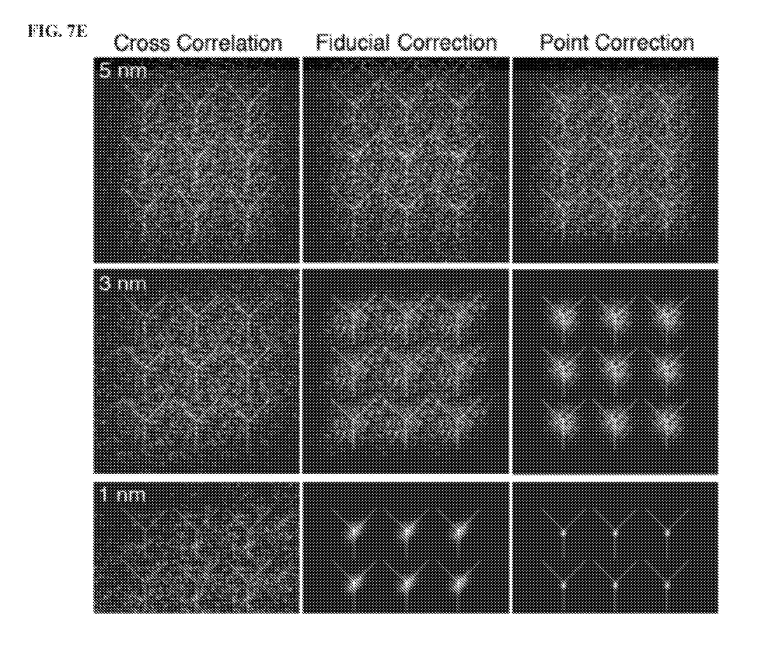

As the precision value for a single localization is a measure of its predicted SEM, we asked whether the distribution of multiple localizations from the same light-emitting source follows a normal distribution. Indeed, the distribution of localizations from a FND closely fit a Gaussian distribution as 71.2% of the distribution fell within 1σ, 96.1% within 2σ, 99.7% within 3σ, (FIG. 9E,F) and as the Gaussian distribution fit was confirmed by the Anderson-Darling test (h=0; FIG. 9G). Based on this, we tested whether the expected SEM value for a single localization correlates with the observed SD of multiple localizations. To do this, FNDs of varying fluorescence intensities were localized and corrected using point correction, and each localized position was binned based on its expected precision value. As shown in FIG. 7A, the observed SD of localizations correlated strongly with their expected SEM (R²=0.97). This correlation was also observed with isolated Alexa-647-conjugated antibody (R²=0.94, FIG. 7B). In addition, multiple localizations from isolated Alexa-647-labeled antibody resulted in a Gaussian distribution similar to FNDs, showing that the normal distribution of localizations around a mean is a general function of SMLM (FIG. 7C, D).

As the center of distribution of multiple localizations represents the best approximate position of the light-emitter, and as the probable distance from the distribution mean for each localization is calculated by the SEM (σ), we propose statistical definitions to differentiate the terms ‘localization precision’ and ‘localization accuracy’. ‘Localization precision’ will be defined as the precision of localizing each individual peak (σ) as calculated by Thompson's equation, and ‘localization accuracy’ will be defined as the probable distance from the distribution mean for each localization. Thus, to obtain ‘localization accuracy’ with 95.5% confidence, ‘localization precision’ needs to be multiplied by 4 (2*2σ) for a single localization (e.g. the fluorescent molecule is 95.5% likely to be located in a 40 nm wide area around a single localized peak of 10 nm precision). While ‘localization accuracy’ can be significantly better when derived from multiple localizations from the same light-emitter (see discussion), for the scope of this paper ‘localization precision’ will be defined as a, and ‘localization accuracy’ 4σ.

### Antibody-Size Limited Accuracy

Accurate localization of single molecules has long been a goal of SMLM. Various statistical approaches have been employed to analyze SMLM images, but they have been restricted to mean estimates of the population, with little insight about the individual molecular structures. While some SMLM studies have achieved molecular level analysis using correlative EM or alignment averaging, they have been limited to structures with known molecular patterns (Sochacki, Shtengel et al. 2014). Given the lack of a proper framework for single molecule imaging and analysis, deciphering the nano-scale organization of heterogeneous structures such as the T cell microcluster has been challenging.

In addition to stage movement correction, a major barrier to accurate localization of single molecules has been inadequate localization precision. To find the minimum precision required for single molecule-level accuracy, FNDs were localized at increasing levels of precision and corrected using the three methods of stage movement correction. The resulting distributions of FND localizations were overlaid with 9 antibodies drawn to scale in a 3×3n grid (12 nm-sized antibodies spaced 12 nm apart) to simulate a densely labeled sample during dSTORM imaging.

At an average precision of 5.4 nm, none of the correction methods allowed discrete visualization of antibodies, as the range of localization distribution (>20 nm) was larger than the antibody size (top row, FIG. 7E, F). At 3.3 nm, the point correction-applied FND localizations began to show discrete visualization of antibody locations, as 95.5% of localizations were distributed within the size of the antibody (2.5 nm, 2.5 nm, [X,Y] SD, Point Correction, middle row, FIG. 7E,F). At 1.0 nm, both fiducial and point correction-applied localizations showed discrete visualization of antibodies, but not cross correlation-applied localizations (bottom row, FIG. 7E). Moreover, we measured sub-nanometer SD in the distribution of point correction-applied localizations (0.5 nm, 0.5 nm, [X,Y] SD; bottom row, FIG. 7E,F), matching the level of precision achieved with feedback loop-based stage drift elimination. These results show two things. First, cross correlation does not achieve sufficient localization accuracy to discriminate between antibody locations, while fiducial correction does so only at very high precision levels. Second, in order to perform dSTORM imaging with antibody-size limited accuracy, the range of localization distribution (i.e. localization accuracy) needs to be smaller than the antibody size. Thus, we chose to use point correction for stage movement correction, and 3 nm as the minimum precision level for dSTORM-localized peaks.

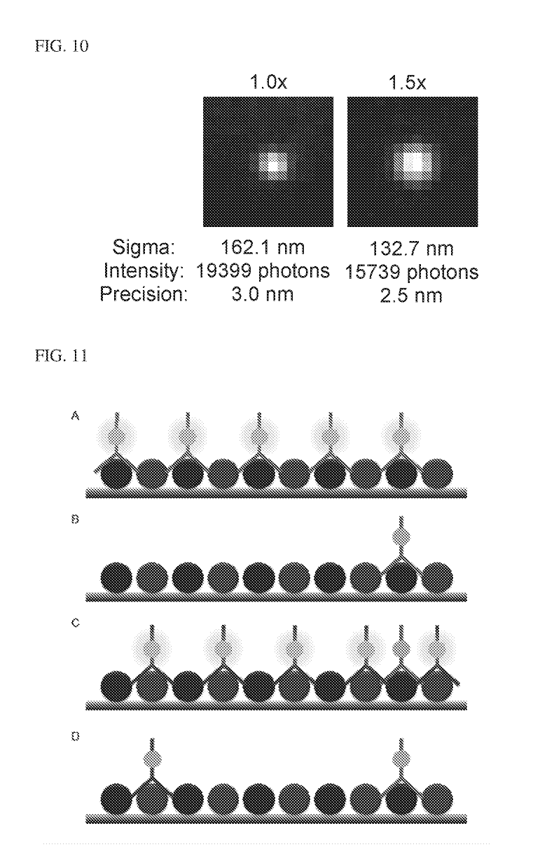

To achieve antibody size-limited accuracy, we sought to optimize localization precision of Alexa-647-labeled antibodies during dSTORM imaging. According to Thomson's equation lower sigma (i.e. pixel size), lower background noise, or higher photon emission can lead to increased precision (Thompson, Larson et al. 2002). Localization of the same FND using different magnification settings showed that addition of 1.5× magnification decreases sigma value and increases precision by 0.5 nm (FIG. 10).

Multiplexed dSTORM

While cellular structures with known molecular patterns (e.g. microtubule, Clathrin-coated pit, nuclear pore complex) have been elegantly characterized using super resolution microscopy techniques, heterogeneous complexes such as the T cell microcluster have been difficult to study due to limits in localization accuracy and multiplexing. The latter, in particular, has been hampered by lack of high-performing SMLM fluorescent reporters, and chromatic aberration and spectral overlap between the fluorescent reporters. To overcome these issues, we have developed a new technique called multiplexed antibody size-limited dSTORM (madSTORM).

In preparation for madSTORM imaging, all antibodies need to be directly conjugated to Alexa-647. Once an Alexa-647-conjugated antibody is bound to the fixed cell sample and imaged using antibody size-limited dSTORM (FIG. 11A), they are unbound using a stripping buffer (FIG. 11B). For any antibody that remains bound, their fluorescence is photo-bleached by exposure to 647 laser in the absence of an oxygen-scavenging solution (FIG. 11B). The cell sample is bound by a new set of Alexa-647-conjugated antibody, imaged, unbound, and photobleached (FIG. 11C,D). These steps can cycle indefinitely, allowing dSTORM imaging of a potentially unlimited number of molecular targets with an antibody size-limited accuracy.

### Multiplexed Super-Resolution View of the Immune Synapse

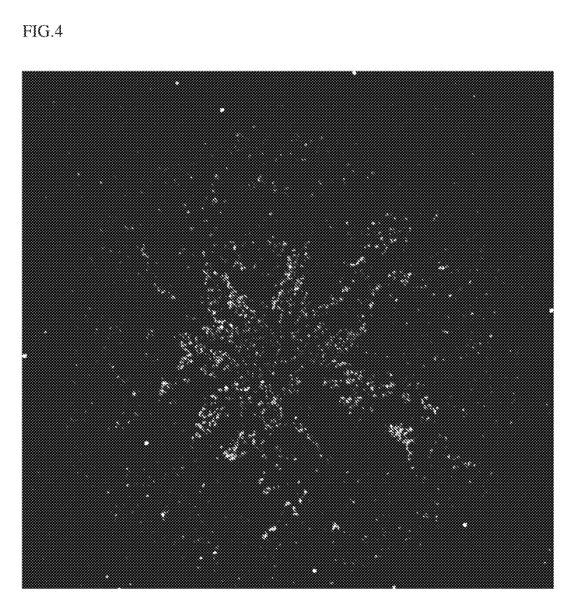

FIG. 4 shows a micrograph of sequential imaging of three T-Cell Receptor micro-complex forming proteins (LAT, SLP76, and pZeta) in a Jurkat T-Cell corrected as described above.

TIRF confocal images were acquired using NIKON Eclipse Ti inverted microscope, 647 nm AOTF modulated LUNB solid state laser (125 mW), 100x SR Apochromat TIRF objective lens (1.49 NA), Andor iXon Ultra 897 EMCCD camera (512×512, 16 μm pixel). dSTORM localization of TIRF confocal images was performed using Thunderstorm plugin (ver. 1.2) on Image J software. Point correction on dSTORM localization data was performed using customized code written on MATLAB software (R2014b).

ALEXA FLUOR-647-labeled antibodies against each protein were sequentially bound, imaged, and rinsed off from the cell. FNDs were simultaneously imaged to eliminate drift during and between imaging each labeled antibody. Each antibody was imaged for 10000 frames at 200 ms exposure for a total imaging duration of 2000 seconds (˜33 minutes). Washing and antibody staining with each labeled antibody required an additional period of ˜30 minutes. In total, imaging of each protein required approximately 70 minutes. In this multiplexed image, average localization precision was 3.61 nm and average alignment precision was 2.07 nm. Thus, drift was reduced to ˜2 nm over ˜3 hours of imaging and mechanical perturbation from repeated washing.

In subsequent super-resolution experiments, data collection of 200 ms per frame for 20 000 frames for a total image collection time of 1.5 hours per antibody has been used. To date, multiplex imaging of 29 separate antibodies corresponding to 29 different proteins has been performed. This involved 43.5 hours of imaging over 10 days. The average alignment error was 2.0 nm among these 29 dSTORM images acquired over the 10 days, which is more than 10 fold improvement over previous multicolor dSTORM images (typically 20-40 nm alignment error) which were typically done with 2-3 colors for shorter durations (<1 hr). These results could not have been obtained without the FND fiducial markers and the tracking procedures that could be implemented due to the optical properties (stability and lifetime) of the FNDs.

The compositions and methods disclosed herein include(s) at least the following embodiments.

### Embodiment 1

A fiducial marker composition comprising a substrate, and a fluorescent nanodiamond immobilized on a surface of the substrate, wherein the substrate and immobilized fluorescent nanodiamond are at least partially top coated with an inert top coating.

### Embodiment 2

The fiducial marker composition of embodiment 1, wherein the fluorescent nanodiamond is immobilized on the substrate with a polymer.

### Embodiment 3

The fiducial marker composition of embodiment 2, wherein the polymer is a charged polymer or a transparent polymer.

### Embodiment 4

The fiducial marker composition of embodiment 3, wherein the charged polymer is polylysine or polyarginine.

### Embodiment 5

A fiducial marker composition comprising a substrate, a transparent polymer immobilized on a surface of the substrate, and a fluorescent nanodiamond embedded in the transparent polymer.

### Embodiment 6

The fiducial marker composition of embodiment 5, further comprising an inert top coating.

### Embodiment 7

The fiducial marker composition of any one of embodiments 1 to 6, wherein the substrate is glass.

### Embodiment 8

The fiducial marker composition of any one of embodiments 1 to 7, wherein the surface is substantially flat.

### Embodiment 9

The fiducial marker composition of any one of embodiments 1 to 8, wherein the density of the fluorescent nanodiamonds on the substrate is between about 10 to about 500 per 100 μm2.

### Embodiment 10

The fiducial marker composition of any one of embodiments 1 to 9, wherein the average largest size of the fluorescent nanodiamond is about 5 nm to about 100 nm.

### Embodiment 11

The fiducial marker composition of any one of embodiments 2 to 10 wherein the polymer is patterned on the substrate surface.

### Embodiment 12

The fiducial marker composition of any one of embodiments 2 to 11, wherein at least two fluorescent nanodiamonds are immobilized in the polymer such that the distance between the at least two fluorescent nanodiamonds and the substrate surface is not identical.

### Embodiment 13

The fiducial marker composition of any one of embodiments 1 to 12, wherein the fluorescent nanodiamond is a multicolor fluorescent nanodiamond.

### Embodiment 14

A method of making a fiducial marker composition comprising immobilizing a fluorescent nanodiamond on a surface of a substrate, and coating the immobilized fluorescent nanodiamond and surface with an inert top coating.

### Embodiment 15

The method of embodiment 14, wherein immobilizing a fluorescent nanodiamond on a surface of a substrate comprises applying a combination comprising the fluorescent nanodiamond and an aqueous solution of a polymer to the surface of the substrate.

### Embodiment 16

The method of embodiment 14, wherein immobilizing a fluorescent nanodiamond on a surface of a substrate comprises coating the surface with a polymer solution; and dispersing the fluorescent nanodiamond onto the polymer coating.

### Embodiment 17

The method of embodiment 16, wherein the polymer coating is patterned before or after dispersing the fluorescent nanodiamond.

### Embodiment 18

The method of any one of embodiments 14 to 17, wherein the substrate is glass.

### Embodiment 19

The method of any one of embodiments 15 to 18, wherein the polymer is a charged polymer or a transparent polymer.

### Embodiment 20

The method of any one of embodiments 14-19, wherein immobilizing a fluorescent nanodiamond on a surface of a substrate comprises functionalizing the surface of the substrate with a functional group that reacts with the fluorescent nanodiamond or a functional group of a functionalized fluorescent nanodiamond; optionally patterning the functionalized surface; and applying a solution comprising the fluorescent nanodiamond or the functionalized fluorescent nanodiamond to the functionalized surface.

### Embodiment 21

The method of embodiment 14, wherein immobilizing a fluorescent nanodiamond on a surface of a substrate comprises immobilizing a pre-formed shape comprising a transparent polymer on the substrate surface, wherein the fluorescent nanodiamond is contained within the object or on a surface of the object.

### Embodiment 22

The composition of any one of embodiments 2-3 and 5 to 12, or the method of any one of embodiments 15-19 and 21, wherein the polymer is a transparent polydimethylsiloxane.

### Embodiment 23

A fiducial marker composition comprising a marker complex comprising a fluorescent nanodiamond and a contrast agent for a nonfluorescent imaging method.

### Embodiment 24

The fiducial marker composition of embodiment 23, wherein the nonfluorescent imaging method is magnetic resonance imaging, computerized tomography imaging, X-ray imaging, or electron microscopy.

### Embodiment 25

The fiducial marker composition of embodiment 23 or 24, wherein the marker complex comprises the fluorescent nanodiamond encapsulated in a liposome, and the liposome further encapsulates the contrast agent.

### Embodiment 26

The fiducial marker composition of embodiment 25 wherein the contrast agent is an osmium-containing moiety.

### Embodiment 27

The fiducial marker composition of embodiment 26, wherein the osmium-containing moiety is osmium tetroxide.

### Embodiment 28

The fiducial marker composition of embodiment 23, wherein the marker complex comprises the fluorescent nanodiamond coupled to a gadolinium-containing moiety, a dysprosium-containing moiety, or a high electron density material.

### Embodiment 29

The fiducial marker composition of embodiment 28, wherein the high electron density material comprises gold, uranium, or tungsten.

### Embodiment 30

The fiducial marker composition of any one of embodiments 23 to 29, wherein the fluorescent nanodiamond is encapsulated in a silica.

### Embodiment 31

An imaging method comprising contacting a sample with the fiducial marker composition of any one of embodiments 1 to 12 or 23 to 30; acquiring a plurality of fluorescent images of a target in the sample and a fluorescent nanodiamond; and correcting a target position in each image by aligning positions of the fluorescent nanodiamond in all images.

### Embodiment 32

The imaging method of embodiment 31, wherein the method comprises a second imaging method.

### Embodiment 33

The imaging method of embodiment 32, wherein the second imaging method is magnetic resonance imaging, computerized tomography imaging, X-ray imaging, or electron microscopy.

### Embodiment 34

The imaging method of embodiment 32, wherein the fluorescent nanodiamond is encapsulated in a liposome, wherein the liposome further encapsulates an osmium-containing moiety.

### Embodiment 35

The imaging method of embodiment 34, wherein the osmium-containing moiety is osmium tetroxide.

### Embodiment 36

The imaging method of embodiment 32, wherein the fluorescent nanodiamond is coupled to a gadolinium-containing moiety, a dysprosium-containing moiety, or a high electron density material.

### Embodiment 37

The imaging method of embodiment 36, wherein the high electron density material comprises gold, uranium, or tungsten.

### Embodiment 38

The imaging method of any one of embodiments 31 to 37, which is a 3-dimensional imaging method.

### Embodiment 39

An imaging method comprising contacting a fiducial marker composition comprising a fluorescent nanodiamond with a sample; acquiring a plurality of fluorescent images, each image comprising a target in the sample and the fluorescent nanodiamond; and correcting a target position in each image by aligning positions of the fluorescent nanodiamond in all images.

### Embodiment 40

The imaging method of embodiment 39, wherein contacting the fluorescent nanodiamond (FND) with the sample comprises binding a functional group on the FND to the sample.

### Embodiment 41

The imaging method of embodiment 39 or 40, wherein the method comprises a second imaging method.

### Embodiment 42

The imaging method of embodiment 41, wherein the second imaging method is magnetic resonance imaging, computerized tomography imaging, X-ray imaging, or electron microscopy.

### Embodiment 43

The imaging method of any one of embodiments 39 to 42, wherein the fluorescent nanodiamond is encapsulated in a liposome, wherein the liposome further encapsulates an osmium-containing moiety.

### Embodiment 44

The imaging method of embodiment 43, wherein the osmium-containing moiety is osmium tetroxide.

### Embodiment 45

The imaging method of any one of embodiments 39 to 44, wherein the fluorescent nanodiamond is coupled to a gadolinium-containing moiety, a dysprosium-containing moiety, or a high electron density material.

### Embodiment 46

The imaging method of embodiment 45, wherein the high electron density material comprises gold, uranium, or tungsten.

### Embodiment 47

The imaging method of any one of embodiments 39 to 46, which is a 3-dimensional imaging method.

### Embodiment 48

The imaging method of any one of embodiments 39 to 47, wherein the fluorescent nanodiamond is encapsulated in silica.

### Embodiment 49

The imaging method of any one of embodiments 31-48, wherein the sample is a solution, a suspension, a cell, a tissue, a cellular membrane, an organelle, or an organism.

### Embodiment 50

A super-resolution imaging correction method comprising determining position coordinates of each of m fluorescent nanodiamonds in each image of a plurality of n images by a Gaussian fitting of the point spread function of each fluorescent nanodiamond in each image, wherein m≥4 and n>1; displacing each image to align the coordinates of a first fluorescent nanodiamond in all images; for each fluorescent nanodiamond other than the first fluorescent nanodiamond, calculating the center of the distribution of positions of the fluorescent nanodiamond over all n displaced images; and displacing each image such that the variance in position of all fluorescent nanodiamond other than the first fluorescent nanodiamond is minimized over all images.

### Embodiment 51

The method of embodiment 50, wherein the imaging method is a 2-dimensional method.

### Embodiment 52

The method of embodiment 50, wherein the imaging method is a 3-dimensional method.

### Embodiment 53

The method of any one of embodiments 50 to 52, wherein the displacing is at least one of a translation, a rotation, or a dilation/contraction.

### Embodiment 54

The method of any one of embodiments 50 to 53, wherein the first fluorescent nanodiamond is the fluorescent nanodiamond with the greatest intensity.

### Embodiment 55

The method of any one of embodiments 50 to 54, wherein displacing each image such that the variance in position of all fluorescent nanodiamonds other than first fluorescent nanodiamond is minimized comprises calculating the mean of the center of the distribution of positions of the fluorescent nanodiamonds over all n displaced images for all fluorescent nanodiamonds other than first fluorescent nanodiamond; calculating the mean position of all fluorescent nanodiamonds other than first fluorescent nanodiamond in each image; and displacing a given image to minimize the difference between the mean of the center of the distribution of positions of the fluorescent nanodiamonds over all n displaced images for all fluorescent nanodiamonds other than first fluorescent nanodiamond and the mean position of all fluorescent nanodiamonds other than first fluorescent nanodiamond in the given image.

In general, the invention may alternatively comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context.

“Or” means “and/or.” The suffix “(s)” as used herein is intended to include both the singular and the plural of the term that it modifies, thereby including one or more of that term (e.g., the film(s) includes one or more films). Reference throughout the specification to “some embodiments”, “another embodiment”, “an embodiment”, and so forth, means that a particular element (e.g., feature, structure, and/or characteristic) described in connection with the embodiment is included in at least some embodiments described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A fiducial marker composition comprising
a substrate, and
a fluorescent nanodiamond immobilized on a surface of the substrate.

2. The fiducial marker composition of claim 1, wherein the fluorescent nanodiamond is immobilized on the substrate with a charged polymer or a transparent polymer.

3. The fiducial marker composition of claim 1, wherein the surface of the substrate is patterned.

4.-5. (canceled)

6. The fiducial marker composition of claim 5, further comprising an inert top coating at least partially coating the substrate and immobilized fluorescent nanodiamond.

7.-13. (canceled)

14. A method of making a fiducial marker composition comprising immobilizing a fluorescent nanodiamond on a surface of a substrate to make the fiducial marker composition of claim 1.

15. The method of claim 14, wherein immobilizing a fluorescent nanodiamond on a surface of a substrate comprises
applying a combination comprising the fluorescent nanodiamond and an aqueous solution of a polymer to the surface of the substrate.

16. The method of claim 14, wherein immobilizing a fluorescent nanodiamond on a surface of a substrate comprises
coating the surface with a polymer solution; and
dispersing the fluorescent nanodiamond onto the polymer coating.

17. The method of claim 14, wherein the surface of the substrate is patterned before or after dispersing the fluorescent nanodiamond.

18.-19. (canceled)

20. The method of claim 14, wherein immobilizing a fluorescent nanodiamond on a surface of a substrate comprises
functionalizing the surface of the substrate with a functional group that reacts with the fluorescent nanodiamond or a functional group of a functionalized fluorescent nanodiamond;
optionally patterning the functionalized surface; and
applying a solution comprising the fluorescent nanodiamond or the functionalized fluorescent nanodiamond to the functionalized surface.

21. The method of claim 14, wherein immobilizing a fluorescent nanodiamond on a surface of a substrate comprises
immobilizing a pre-formed shape comprising a transparent polymer on the substrate surface, wherein the fluorescent nanodiamond is contained within the object or on a surface of the object.

22. (canceled)

23. The fiducial marker composition of claim 1, wherein
the fluorescent nanodiamond is in a complex with a contrast or imaging agent for a nonfluorescent imaging method.

24.-38. (canceled)

39. An imaging method comprising
contacting the fiducial marker composition of claim 1 with a sample;
acquiring a plurality of fluorescent images, each image comprising a target in the sample and the fluorescent nanodiamond; and
correcting a target position in each image by aligning positions of the fluorescent nanodiamond in all images.

40. (canceled)

41. The imaging method of claim 39, wherein the method comprises a second imaging method.

42. The imaging method of claim 41, wherein the second imaging method is magnetic resonance imaging, computerized tomography imaging, X-ray imaging, or electron microscopy.

43.-46. (canceled)

47. The imaging method of claim 39, which is a 3-dimensional imaging method.

48. (canceled)

49. The imaging method of claim 39, wherein the sample is a solution, a suspension, a cell, a tissue, a cellular membrane, an organelle, or an organism.

50. A super-resolution imaging correction method comprising
determining position coordinates of each of m fluorescent nanodiamonds in each image of a plurality of n images by Gaussian fitting of the point spread function of each fluorescent nanodiamond in each image, wherein m≥4 and n>1;
displacing each image to align the coordinates of a first fluorescent nanodiamond in all images;
displacing each image such that the variance in position of all fluorescent nanodiamond other than the first fluorescent nanodiamond is minimized over all images.

51. The method of claim 50, wherein the imaging method is a multi-modal method.

52. The method of claim 50, wherein the imaging method is a 3-dimensional method.

53. The method of claim 50, wherein the displacing is at least one of a translation, a rotation, or a dilation/contraction.

54.-55. (canceled)
